(19) United States
(12) Patent Application Publication
(10) Pub. No.: US 2025/0266175 A1
(43) Pub. Date: Aug. 21, 2025

(54) SYSTEM AND METHOD FOR PATIENT-CENTRIC GENERATIVE PATIENT REPORTED OUTCOMES

(71) Applicant: GE Precision Healthcare LLC, Waukesha, WI (US)

(72) Inventors: Akshit Achara, Bangalore (IN); Anuradha Kanamarlapudi, Bangalore (IN); Sanand Sasidharan, Bangalore (IN); Sai Vivek Velkuru, Bangalore (IN)

(21) Appl. No.: 18/581,114

(22) Filed: Feb. 19, 2024

(51) Int. Cl. G16H 80/00; G16H 10/20; G16H 10/60

(52) U.S. Cl. CPC G16H 80/00; G16H 10/20; G16H 10/60

(57) ABSTRACT

A remote patient monitoring system includes a memory encoding processor-executable routines. The remote patient monitoring system also includes a processing system configured to access the memory and to execute the processor-executable routines, wherein the routines, when executed by the processing system, cause the processing system to perform actions. The actions include obtaining feedback from one or more sensors, wherein the feedback relates to one or more health parameters of a subject. The actions also include obtaining past medical records of the subject. The actions further include obtaining a clinical guideline specific to a condition of the subject. The actions even further include generating, via a question generation module, a personalized patient reported outcome questionnaire based at least on the feedback, the past medical records of the subject, and the clinical guideline, wherein the personalized patient reported outcome questionnaire is compatible with the clinical guideline.

### BACKGROUND

The subject matter disclosed herein relates to a system and method for patient-centric generative patient reported outcomes.

Sometimes patients are recommended for remote patient monitoring after medical practitioners (e.g., oncologists) perform a risk assessment for a medical illness, disease, or condition after surgery or treatment (e.g., cancer treatment). The patient monitoring can be both objective and subjective. The subjective component includes sending questionnaires to patients to assess their quality of life and to determine the next steps during the course of their treatment. The current mechanism for remote patient monitoring includes static questionnaires that (in the case of cancer) are predetermined based on the cancer type of a patient or the questionnaire is designed by the oncologist specifically for the patient. These static questionnaires are limited in the parameters that are considered (e.g., limited to 1 or 2 parameters) which may impact the effectives of the remote monitoring. In addition, it may be difficult to keep up with changes or updates in guidelines for remote monitoring for the particular illness, disease, or condition. The manual generation of these questionnaires for each patient is a cumbersome as many reports and other data need to be looked at. Thus, the process of manually generating these questionnaires may substantially burden the medical practitioners and the medical facility (e.g., hospital, clinics, etc.).

### BRIEF DESCRIPTION

A summary of certain embodiments disclosed herein is set forth below. It should be understood that these aspects are presented merely to provide the reader with a brief summary of these certain embodiments and that these aspects are not intended to limit the scope of this disclosure. Indeed, this disclosure may encompass a variety of aspects that may not be set forth below.

In one embodiment, a remote patient monitoring system is provided. The remote patient monitoring system includes a memory encoding processor-executable routines. The remote patient monitoring system also includes a processing system configured to access the memory and to execute the processor-executable routines, wherein the routines, when executed by the processing system, cause the processing system to perform actions. The actions include obtaining feedback from one or more sensors, wherein the feedback relates to one or more health parameters of a subject. The actions also include obtaining past medical records of the subject. The actions further include obtaining a clinical guideline specific to a condition of the subject. The actions even further include generating, via a question generation module, a personalized patient reported outcome questionnaire based at least on the feedback, the past medical records of the subject, and the clinical guideline, wherein the personalized patient reported outcome questionnaire is compatible with the clinical guideline.

In another embodiment, a computer-implemented method for remote patient monitoring is provided. The computer-implemented method includes obtaining, at a processing system, feedback from one or more sensors, wherein the feedback relates to one or more health parameters of a subject. The computer-implemented method also includes obtaining, via the processing system, past medical records of the subject. The computer-implemented method further includes obtaining, via the processing system, a clinical guideline specific to a condition of the subject. The computer-implemented method even further includes generating, via a question generation module of the processing system, a personalized patient reported outcome questionnaire based at least on the feedback, the past medical records of the subject, and the clinical guideline, wherein the personalized patient reported outcome questionnaire is compatible with the clinical guideline.

In a further embodiment, a non-transitory computer-readable medium, the computer-readable medium including processor-executable code that when executed by a processing system, causes the processing system to perform actions. The actions include obtaining feedback from one or more sensors, wherein the feedback relates to one or more health parameters of a subject. The actions also include obtaining past medical records of the subject. The actions further include obtaining a clinical guideline specific to a condition of the subject. The actions even further include generating, via a question generation module, a personalized patient reported outcome questionnaire based at least on the feedback, the past medical records of the subject, and the clinical guideline, wherein the personalized patient reported outcome questionnaire is compatible with the clinical guideline.

### BRIEF DESCRIPTION OF THE DRAWINGS

These and other features, aspects, and advantages of the present subject matter will become better understood when the following detailed description is read with reference to the accompanying drawings in which like characters represent like parts throughout the drawings, wherein:

FIG. 1 is a block diagram of a system for generating patient-centric patient reported outcomes, in accordance with aspects of the present disclosure;

FIG. 2 is a schematic diagram of a plurality of modules to be utilizing by a remote patient monitoring system, in accordance with aspects of the present disclosure;

FIG. 3 is a schematic diagram of an architecture for the system in FIG. 1, in accordance with aspects of the present disclosure;

FIG. 4 is a flow chart of a method for generating a personalized patient reported outcome questionnaire, in accordance with aspects of the present disclosure;

FIG. 5 is a flow chart of a method for processing responses to a personalized patient reported outcome questionnaire, in accordance with aspects of the present disclosure;

FIG. 6 is a flow chart of a method for corroborating responses to a personalized patient reported outcome questionnaire, in accordance with aspects of the present disclosure;

FIG. 7 is a flow chart of a method for generating a personalized patient reported outcome questionnaire report, in accordance with aspects of the present disclosure;

FIG. 8 is a schematic diagram of a personalized patient reported outcome questionnaire report, in accordance with aspects of the present disclosure;

FIG. 9 is a flow chart of a method for utilizing clinician edits and feedbacks, in accordance with aspects of the present disclosure; and

FIG. 10 is a schematic diagram of an example processor platform for a portable computing device to be utilized with the disclosed techniques.

### DETAILED DESCRIPTION

One or more specific embodiments will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

Some generalized information is provided to provide both general context for aspects of the present disclosure and to facilitate understanding and explanation of certain of the technical concepts described herein.

The term processor, processing system, or processing unit, as used herein, refers to any type of processing unit that can carry out the required calculations needed for the various embodiments, such as single or multi-core: CPU, Accelerated Processing Unit (APU), Graphics Board, DSP, FPGA, ASIC or a combination thereof.

As used herein, the term “computing system” refers to an electronic computing device such as, but not limited to, a single computer, virtual machine, virtual container, host, server, laptop, and/or mobile device, or to a plurality of electronic computing devices working together to perform the function described as being performed on or by the computing system. As used herein, the terms “application”, “application module” (or “module”), “engine”, or “program”, or “plugin” refers to one or more sets of computer software instructions (e.g., computer programs and/or scripts) executable by one or more processors of a computing system to provide particular functionality. Computer software instructions can be written in any suitable programming languages, such as C, C++, C#, Pascal, Fortran, Perl, MATLAB, SAS, SPSS, JavaScript, AJAX, and JAVA. Such computer software instructions can comprise an independent application with data input and data display aspects (e.g., modules). Alternatively, the disclosed computer software instructions can be classes that are instantiated as distributed objects. The disclosed computer software instructions can also be component software, for example JAVABEANS or ENTERPRISE JAVABEANS. Additionally, the disclosed applications or engines can be implemented in computer software, computer hardware, or a combination thereof.

As used herein, the terms “automatic” and “automatically” refer to actions that are performed by a computing device or computing system (e.g., of one or more computing devices) without human intervention. For example, automatically performed functions may be performed by computing devices or systems based solely on data stored on and/or received by the computing devices or systems despite the fact that no human users have prompted the computing devices or systems to perform such functions. As but one non-limiting example, the computing devices or systems may make decisions and/or initiate other functions based solely on the decisions made by the computing devices or systems, regardless of any other inputs relating to the decisions.

The present disclosure provides for systems and methods for remote patient monitoring In particular, the disclosed systems and methods provide for a guideline compatible questionnaire tool configured to automatically generate relevant questionnaires for a patient (e.g., subject) given based on (but limited to) patient history, objective monitoring, and previously answered questionnaires by the patient and other similar patients.

The disclosed systems and methods include obtaining feedback from one or more sensors, wherein the feedback relates to one or more health parameters of a subject. The disclosed systems and methods also include obtaining past medical records of the subject. The disclosed systems and methods further include obtaining a clinical guideline specific to a condition of the subject. The disclosed systems and methods even further include generating, via a question generation module, a personalized patient reported outcome questionnaire based at least on the feedback, the past medical records of the subject, and the clinical guideline, wherein the personalized patient reported outcome questionnaire is compatible with the clinical guideline.

In certain embodiments, the disclosed systems and methods include obtaining questionnaires of other subjects with a similar medical history to the subject. In certain embodiments, the disclosed systems and methods also include generating, via the question generation module, the personalized patient reported outcome questionnaire based at least on the feedback, the past medical records of the subject, the clinical guideline, and the questionnaires of the other subjects.

In certain embodiments, the disclosed systems and methods include obtaining, at a response understanding module, one or more responses from the subject to one or more questions from the personalized patient reported outcome questionnaire. In certain embodiments, the disclosed systems and methods also include corroborating, via a response corroboration module, the one or more responses with past responses to questions by the subject, the feedback, and the past medical records of the subject. In certain embodiments, the disclosed systems and methods further include updating, via the question generation module, a remaining portion of the personalized patient reported outcome questionnaire based on a level of corroboration between the one or more responses with the past responses to questions by the subject, the feedback, and the past medical records of the subject.

In certain embodiments, the disclosed systems and methods include notifying, via the response corroboration module, the question generation module when the one or more responses are not consistent with the past responses to the questions by the subject, the feedback, and/or the past medical records of the subject. In certain embodiments, the disclosed systems and methods also include generating, via the question generation module, one or more follow-up questions to clarify any inconsistency when the one or more responses are not consistent with the past responses to the questions by the subject, the feedback, and/or the past medical records of the subject.

In certain embodiments, the disclosed systems and methods include obtaining, at a response understanding module, one or more responses from the subject to one or more questions from the personalized patient reported outcome questionnaire, wherein the one or more responses are outside a scope of the personalized patient reported outcome questionnaire but relevant to subsequent clinical decisions. In certain embodiments, the disclosed systems and methods also include generating, via the question generation module, one or more follow-up questions based on the one or more responses. In certain embodiments, the disclosed systems and methods include analyzing, via the response understanding module, the one or more responses from the subject utilizing a natural language understanding model to determine any facts outside the scope of the personalized patient reported outcome questionnaire but relevant to subsequent clinical decisions.

In certain embodiments, the disclosed systems and methods include receiving, at a report generation module, an entirety of responses from the subject to the personalized patient reported outcome questionnaire. In certain embodiments, the disclosed systems and methods also include analyzing, via the report generation module, the entirety of the responses from the subject in the context of both the past medical records of the subject and the clinical guideline. In certain embodiments, the disclosed systems and methods further include generating, via the report generation module, a personalized patient reported outcome questionnaire report based on the analysis, wherein facts within the personalized patient reported outcome questionnaire report are ranked in terms of relevancy and novelty and presented in a concise manner.

In certain embodiments, the disclosed systems and methods include logging, via a usage and feedback logging module, edits to the personalized patient reported outcome questionnaire report. In certain embodiments, the disclosed systems and methods also include providing, via the usage and feedback logging module, the edits to a finetuning layer of a language model utilized by the question generation module in generating questions for subsequently generated personalized patient reported outcome questionnaires.

The disclosed embodiments provide for the digitization of guidelines (e.g., graphically) to enable the following of patient over time with respect to a condition. The disclosed embodiments enable the digitized guidelines to adapt to any changes in the structure which enables the clinician (e.g. oncologist) keep up with the latest updates in the guidelines without any effort from their end. The disclosed embodiments help find similar patients and questionnaires using patient histories and enable generating similar questionnaires for the patient of interest. The disclosed embodiments enable all elements of a patient's journey (and similar patient journeys) to be considered. The disclosed embodiments enable more accurate questionnaires to be sent to remote patients using an algorithm that takes into account the rich patient data and history. The disclosed embodiments enable the improvement in access and speed and delivery of care to the patient. The disclosed embodiments improve the efficiency and accuracy of remote patient monitoring and, thus, the quality of life of the patient. The disclosed embodiments reduce the burden on healthcare facilities and clinicians in generating the questionnaires. The disclosed embodiments also reduce cost saving across healthcare.

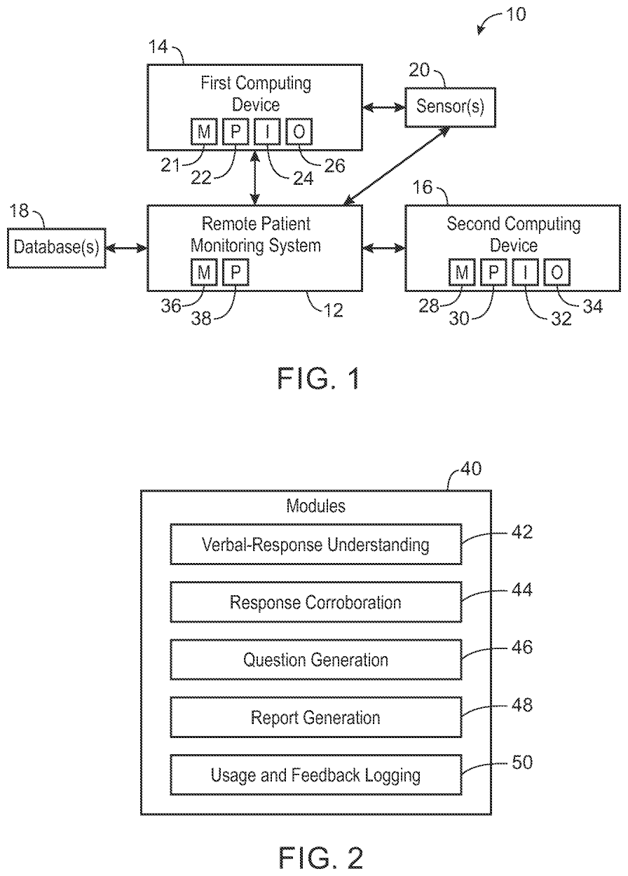

With the preceding in mind, and by way of providing useful context, FIG. 1 depicts is a block diagram of a system 10 for generating patient-centric patient reported outcomes. The system 10 may be utilized to follow a patient (e.g., subject) after any medical treatment or surgery in accordance with clinical guidelines specific to the condition (e.g., type of disease, type of cancer, type of surgery, type of treatment, etc.) of the subject. The system 10 includes a remote patient monitoring system 12 communicatively coupled to a first computing device 14, a second computing device 16, one or more databases 18, and one or more sensors 20.

The first computing device 14 (e.g., utilized by a patient (e.g., subject) includes a memory 21 and a processing system 22 (e.g., one or more processors). The first computing device 14 may be a laptop, tablet, smartphone, or other type of computing device. The first computing device 14 includes one or more input devices 24 (e.g., keyboard, touchscreen, microphone, etc.) to enable the patient to communicate with the remote patient monitoring system 12. For example, the patient may answer questions for a patient reported outcome questionnaire (e.g., personalized patient reported outcome questionnaire) via the input devices 24. The first computing device 14 also include or more output devices 26 (e.g., display, speaker, etc.).

In certain embodiments, the first computing device 14 may be in communication with the one or more sensors 20 coupled to and/or utilized on the patient to obtain feedback related to one or more health parameters or physiological parameters of the patient. The one or more sensors 20 are configured to measure these health parameters or physiological parameters. These parameters may include but are not limited blood pressure, temperature, glucose levels, oxygen saturation, respiration rate, heart rate, and/or any other parameter. The one or more sensors 20 may include but are not limited to a glucose monitor, a pulse oximeter, a heart rate monitor, or another type of sensor. The first computing device 14 may communicate the feedback from the sensors 20 to the remote patient monitoring system 12. In certain embodiments, the one or more sensors 20 may additionally or alternatively communicate the feedback directly to the remote patient monitoring system 12.

The second computing device 16 (e.g., utilized by medical practitioner or clinician (e.g., oncologist)) includes a memory 28 and a processing system 30 (e.g., one or more processors). The second computing device 16 may be a laptop, tablet, smartphone, or other type of computing device. The second computing device 16 includes one or more input devices 32 (e.g., keyboard, touchscreen, microphone, etc.) to enable the medical practitioner to communicate with the remote patient monitoring system 12. For example, the medical practitioner may obtain a patient reported outcome questionnaire (e.g., personalized patient reported outcome questionnaire) and edit and/or provide feedback for the patient reported outcome questionnaire. The second computing device 16 also include or more output devices 34 (e.g., display, speaker, etc.).

The remote patient monitoring system 12 includes a memory 36 and a processing system 38 (e.g., one or processors). The processing system 38 is configured to execute a plurality of modules stored within the memory 36 to perform a number of actions related to the patient-centric generative patient reported outcome questionnaires. The remote patient monitoring system 12 is configured to access data from the one or more databases 18. The one or more databases 18 may include a patient database for storing past medical records of patients, patient profiles, previous responses to past patient reported outcome questionnaires, past patient reported outcome questionnaires for different types of patient profiles, and/or other patient specific data. In certain embodiments, the patient database may include a structural database that includes a number of parameters related to the patient (e.g., including complaint parameters, diagnosis parameters, and/or treatment parameters). In certain embodiments, the one or more databases 18 may store one or more natural language understanding models to help in analyzing and understanding patient responses to patient reported outcome questionnaires. In certain embodiments, the one or more databases 18 may store medical ontologies (e.g., for different areas in the medial field) to also help in analyzing and understanding patient responses to patient reported outcome questionnaires. In certain embodiments, the one or more databases 18 may store different types of clinical guidelines for a variety of different patient conditions. In certain embodiments, the one or more databases 18 may store language models to be utilized in generating questions for patient reported outcome questionnaires (and/or rephrasing questions).

The remote patient monitoring system 12 is also configured to obtain feedback from the one or more sensors 20 (e.g., coupled to the patient). The remote patient monitoring system 12 is further configured to obtain/receive past medical records of the patient (e.g., patient profile, past complaint parameters, past diagnostic parameters, past treatment parameters, previous responses to patient reported outcome questionnaires, etc.). The remote patient monitoring system 12 is further configured to obtain/receive a clinical guideline specific to a condition of the patient. An example of guideline is the National Comprehensive Cancer Network® guidelines in oncology for different cancer types. The remote patient monitoring system 12 is even further configured to generate a personalized patient reported outcome questionnaire (e.g., compatible with clinical guideline) based at least on the feedback from the sensors 20, the past medical records of the patient, and the obtained clinical guideline specific to the condition of the patient.

In certain embodiments, the remote patient monitoring system 12 is configured to obtain patient reported outcome questionnaires of other subjects with a similar medical history or patient profile to the patient. The remote patient monitoring system 12 may also be configured to generate the personalized patient reported outcome questionnaire utilizing one or more of these other questionnaires in conjunction with the feedback, the past medical records of the subject, and the obtained clinical guideline specific to the condition of the patient.

The remote patient monitoring system 12 is configured to provide questions from the personalized patient reported outcome questionnaire to the patient on the first computing device 14 and to receive and to receive and to interpret (e.g., understand) the patient's answers to the questions.

The remote patient monitoring system 12 is also configured to obtain/receive the responses from the patient to questions of the personalized patient reported outcome questionnaire and to corroborate the responses with past responses to questions by the patient, the feedback from the sensors 20, and the past medical records of the subject. In certain embodiments, the remoter patient monitoring system 12 is configured to update the remaining portion of the personalized patient reported outcome questionnaire based on a level of corroboration between the present responses and the past responses, the feedback, and the past medical records of the subject.

In certain embodiments, the remote patient monitoring system 12 is configured to notify a question generation module when one or more of the present responses are not consistent with the past responses to the questions, the feedback, and/or the past medical records of the subject. In certain embodiments, the remote patient monitoring system 12 is configured to generate one or more follow-up questions to clarify any inconsistency when one or more of the present responses are not consistent with the past responses to the questions, the feedback, and/or the past medical records of the subject.

In certain embodiments, the obtained responses from the patient may be outside a scope of the personalized patient reported outcome questionnaire but relevant to subsequent clinical decisions. In this scenario, the remote patient monitoring system 12 is configured to generate one or more follow-up questions based on these responses outside the scope. In certain embodiments, the remote patient monitoring system 12 may analyze one or more responses form the subject utilizing a natural language understanding model (and the medical ontologies) to determine any facts outside the scope of the personalized patient reported outcome questionnaire but relevant to subsequent clinical decisions.

The remote patient monitoring system 12 is also configured to receive an entirety of responses from the patient to the personalized patient reported outcome questionnaire and to analyze the entirety of the responses from the subject in the context of both the past medical records of the patient and the obtained clinical guideline. The remote patient monitoring system 12 is configured to generate a personalized patient reported outcome questionnaire report based on this analysis. In particular, the remote patient monitoring system is configured to rank facts within the report in terms of relevancy and novelty and to present the facts in a concise manner. The most relevant and novel facts are presented at the beginning of the report.

The remote patient monitoring system 12 is further configured to log any edits of the report and/or feedback to the report. The remote patient monitoring system 12 is configured to provide the edits and/or feedback to a finetuning layer of a language model utilized by a question generation module in generating questions for subsequently generate personalized patient reported outcome questionnaires.

FIG. 2 is a schematic diagram of a plurality of modules 40 to be utilizing by a remote patient monitoring system 12 in FIG. 1. The modules 40 include a verbal-response understanding module 42 (e.g., response understanding module), a response corroboration module 44, a question generation module 46, a report generation module 48, and a usage and feedback logging module 50. In certain embodiments, other types of modules 40 may be utilized by the remote patient monitoring system 12.

The verbal-response understanding module 42 is configured to receive and to interpret (e.g., determine) the responses to questions from the patient. The verbal-response understanding module 42 is configured to receive/obtain the answers. The verbal-response understanding module 42 is also configured to obtain and to utilize one or more natural language understanding models and medical ontologies in interpreting the responses to questions from the patient. The verbal-response understanding module 42 provides the ability to record and follow-up on patient responses outside the scope of the current questionnaire but that may have some significance to future clinical decisions. For example, if a patient gives additional details to their answer (e.g., “I do not have shortness of breath, but have a pain while breathing), then these additional details need to be followed-up with appropriate questions and the responses recorded. The verbal-response understanding module 42 is configured to analyze the one or more responses from the subject utilizing a natural language understanding model to determine any facts outside the scope of the personalized patient reported outcome questionnaire but relevant to subsequent clinical decisions. The verbal-response understanding module 42 is configured to communicate with the response corroboration module 44.

The response corroboration module 44 is configured to obtain the responses to the questions from the questionnaire from the verbal-response understanding module 42. The response corroboration module 44 is also configured to obtain/access previous responses to the questionnaire by the patient and the medical records of the patient (e.g., patient profile, past complaint parameters, past diagnostic parameters, etc.). The response corroboration module 44 is further configured to obtain the feedback from sensors of health or physiological parameters of the patient. The response corroboration module 44 is also configured to corroborate the responses with past responses to questions by the patient, the feedback from the sensors, and the past medical records of the subject. In certain embodiments, the response corroboration module 44 is configured to update the remaining portion of the personalized patient reported outcome questionnaire based on a level of corroboration between the present responses and the past responses, the feedback, and the past medical records of the subject.

In certain embodiments, the response corroboration module 44 is configured to notify a question generation module when one or more of the present responses are not consistent with the past responses to the questions, the feedback, and/or the past medical records of the subject. In certain embodiments, the response corroboration module 44 is configured to causes the question generation module 46 to generate one or more follow-up questions to clarify any inconsistency when one or more of the present responses are not consistent with the past responses to the questions, the feedback, and/or the past medical records of the subject. The response corroboration module 44 is configured to communicate with the question generation module 46.

The question generation module 46 is configured to obtain configured to obtain feedback from the one or more sensors (e.g., coupled to the patient). The question generation module 46 is further configured to obtain/receive past medical records of the patient (e.g., patient profile, past complaint parameters, past diagnostic parameters, past treatment parameters, previous responses to patient reported outcome questionnaires, etc.). The question generation module 46 is further configured to obtain/receive a clinical guideline specific to a condition of the patient. An example of guideline is the National Comprehensive Cancer Network® guidelines in oncology for different cancer types. The question generation module 46 is even further configured to generate a personalized patient reported outcome questionnaire (e.g., compatible with clinical guideline) based at least on the feedback from the sensors, the past medical records of the patient, and the obtained clinical guideline specific to the condition of the patient. The question generation module 46 is also configured to obtain one or more language models and to utilize the one or more language models in generating the questions for the personalized patient reported outcome questionnaire.

In certain embodiments, the question generation module 46 is configured to obtain patient reported outcome questionnaires of other subjects with a similar medical history or patient profile to the patient. The question generation module 46 may also be configured to generate the personalized patient reported outcome questionnaire utilizing one or more of these other questionnaires in conjunction with the feedback, the past medical records of the subject, and the obtained clinical guideline specific to the condition of the patient.

In certain embodiments, the question generation module 46 is configured to generate one or more follow-up questions (or clarifying questions) to clarify any inconsistency (e.g., as determined by the response corroboration module 44) when one or more of the present responses are not consistent with the past responses to the questions, the feedback, and/or the past medical records of the subject. For example, a patient's answer may not be consistent with the sensor readings (e.g., heart rate or respiratory rate), or previous answers, the inconsistency needs to be resolved then and there via a clarifying question. In certain embodiments, the obtained responses from the patient may be outside a scope of the personalized patient reported outcome questionnaire but relevant to subsequent clinical decisions. In this scenario, the question generation module 46 is configured to generate one or more follow-up questions based on these responses outside the scope. For example, if a patient provides additional details in their answer (e.g., “I do not have shortness of breath, but have a pain while breathing”), that information needs to be followed-up with appropriate questions and to be recorded. The question generation module 46 is configured to provide the questions from the personalized patient reported outcome questionnaire to a patient via a user interface on a computing device utilized by the patient. The question generation module 46 is also configured to communicate with the report generation module 48.

In generating questions, the question generation module 46 is configured to analyze the feedback from the sensors, the past medical history of the patient, and the previous responses to questions to access the necessity of each of the subsequent questions. Thus, unnecessary questions, repeated questions, and questions lacking an empathetic tone can be avoided. For example, if a patient has an elevated respiratory rate or heart palpitations (which can be detected via the sensors), the questions on heart palpitations and shortness of breath can be avoided.

The report generation module 48 is configured to obtain/receive an entirety of responses from the patient to the personalized patient reported outcome questionnaire. The report generation module 48 is also configured to obtain/receive past medical records of the patient (e.g., patient profile, past complaint parameters, past diagnostic parameters, past treatment parameters, previous responses to patient reported outcome questionnaires, etc.). The report generation module 48 is further configured to obtain/receive a clinical guideline specific to a condition of the patient. The report generation module 48 is to analyze the entirety of the responses from the subject in the context of both the past medical records of the patient and the obtained clinical guideline. The report generation module 48 is configured to generate a personalized patient reported outcome questionnaire report based on this analysis. In particular, the report generation module 48 is configured to rank facts within the report in terms of relevancy and novelty and to present the facts in a concise manner. The most relevant and novel facts are presented at the beginning of the report. For example, when answers are beyond an expected range, or pertains to a new symptom or adverse event, can be highlighted. The report generation module 48 is configured to provide the report to a clinician via a user interface on a computing device utilized by the clinician. The report generation module 48 is also configured to obtain user profile and user preferences for the clinician and to utilize these in generating the report.

The usage and feedback logging module 50 is configured to receive and to log any edits of the report and/or feedback to the report provided by the clinician. The usage and feedback logging module 50 is configured to provide the edits and/or feedback to a finetuning layer of a language model utilized by the question generation module 46 in generating questions for subsequently generate personalized patient reported outcome questionnaires. This enables accounting for any changes in language specific to a region. In particular, certain terms can be replaced with more regionally understood equivalent terms. The usage and feedback logging module 50 enables automatic learning from a doctor's, hospital's, or region's style of asking questions.

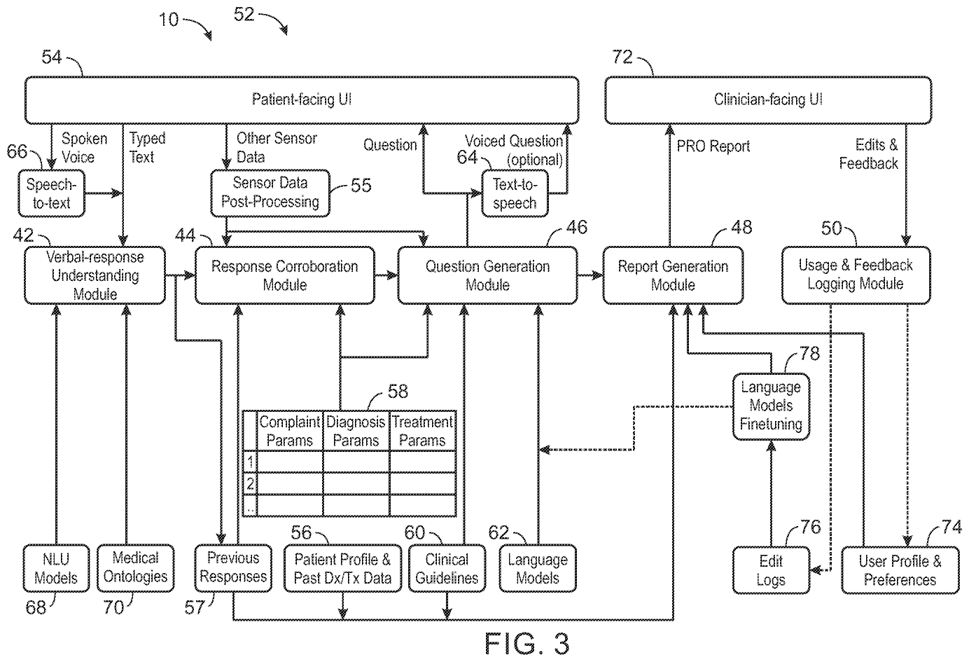

FIG. 3 is a schematic diagram of an architecture 52 for the system 10 in FIG. 1. The question generation module 46 obtains feedback from the one or more sensors (e.g., coupled to the patient) via a user interface 54 (e.g., patient-facing user interface) utilized by the patient on a computing device utilized by the patient. The sensor data may be post-processed prior to being provided to the response corroboration module 44 and the question generation module 46 as indicated by reference number 55. The question generation module 46 is further configured to obtain/receive past medical records 56 of the patient (e.g., patient profile, past complaint parameters, past diagnostic parameters, past treatment parameters, previous responses to patient reported outcome questionnaires, 57 etc.). The past medical records 56 may include a structured database 58 (e.g., having past complaint parameters, past diagnostic parameters, and past treatment parameters). The question generation module 46 further obtains a clinical guideline 60 specific to a condition of the patient. The question generation module 46 generates a personalized patient reported outcome questionnaire (e.g., compatible with clinical guideline 60) based at least on the feedback from the sensors, the past medical records 56 of the patient, and the obtained clinical guideline 60 specific to the condition of the patient. The question generation module 46 also obtains one or more language models 62 and utilizes the one or more language models 62 in generating the questions for the personalized patient reported outcome questionnaire. The question generation module 46 communicates with the report generation module 48.

In certain embodiments, the question generation module 46 obtains patient reported outcome questionnaires of other subjects with a similar medical history or patient profile to the patient. The question generation module 46 may also generate the personalized patient reported outcome questionnaire utilizing one or more of these other questionnaires in conjunction with the feedback, the past medical records 56 of the subject, and the obtained clinical guideline 60 specific to the condition of the patient.

In certain embodiments, the questions from the personalized patient reported outcome questionnaire are provide in a textual form to the patient via the user interface 54. In certain embodiments, the questions from the personalized patient reported outcome questionnaire are converted from text to speech as indicated by reference numeral 64. Thus, in certain embodiments, the questions from the personalized patient reported outcome questionnaire are verbally provided (e.g., via a speaker) to the patient.

In certain embodiments, the responses or answers to the questions from the personalized patient reported outcome questionnaire are provided in a textual form via the user interface 54 to the verbal-response understanding module 42. In certain embodiments, the response or answers to the questions are provided in a verbal form (e.g., via a microphone) via the user interface 54, converted from speech to text as indicated by reference numeral 66, and provided to the verbal-response understanding module 42.

The verbal-response understanding module 42 receives and interprets the answers or responses to questions from the patient. The verbal-response understanding module 42 obtains and utilizes one or more natural language understanding models 68 and medical ontologies 70 in interpreting the responses to questions from the patient. The verbal-response understanding module 42 may analyze the one or more responses from the patient utilizing a natural language understanding model 68 to determine any facts outside the scope of the personalized patient reported outcome questionnaire but relevant to subsequent clinical decisions. The verbal-response understanding module 42 communicate with the response corroboration module 44.

The response corroboration module 44 obtains the responses to the questions from the questionnaire from the verbal-response understanding module 42. The response corroboration module 44 is also obtains/accesses previous responses 57 to the questionnaire by the patient and the medical records 56 of the patient (e.g., patient profile, past complaint parameters, past diagnostic parameters, etc.). The response corroboration module 44 further obtains the feedback from sensors of health or physiological parameters of the patient. The response corroboration module 44 also corroborates the responses with past responses 57 to questions by the patient, the feedback from the sensors, and the past medical records 56 of the subject. In certain embodiments, the response corroboration module 44 updates the remaining portion of the personalized patient reported outcome questionnaire based on a level of corroboration between the present responses and the past responses 57, the feedback, and the past medical records 56 of the patient.

In certain embodiments, the response corroboration module 44 notifies the question generation module 46 when one or more of the present responses are not consistent with the past responses 57 to the questions, the feedback, and/or the past medical records 56 of the patient. In certain embodiments, the response corroboration module 44 causes the question generation module 46 to generate one or more follow-up questions to clarify any inconsistency when one or more of the present responses are not consistent with the past responses to the questions, the feedback, and/or the past medical records of the subject. The response corroboration module 44 communicates with the question generation module 46.

The report generation module 48 obtains an entirety of responses from the patient to the personalized patient reported outcome questionnaire (which are now part of the previous responses 57). The report generation module 48 also obtains past medical records 56 the patient (e.g., patient profile, past complaint parameters, past diagnostic parameters, past treatment parameters, previous responses to patient reported outcome questionnaires, etc.). The report generation module 48 further obtains the clinical guideline 60 specific to a condition of the patient. The report generation module 48 analyzes the entirety of the responses from the subject in the context of both the past medical records 56 of the patient and the obtained clinical guideline 60. The report generation module 48 generates a personalized patient reported outcome questionnaire report based on this analysis. In particular, the report generation module 48 is configured to rank facts within the report in terms of relevancy and novelty and to present the facts in a concise manner. The most relevant and novel facts are presented at the beginning of the report. For example, when answers are beyond an expected range, or pertains to a new symptom or adverse event, can be highlighted. The report generation module 48 provides the report to a clinician via a user interface 72 on a computing device utilized by the clinician. The report generation module 48 also obtains user profile and user preferences 74 for the clinician and utilizes these in generating the report.

The usage and feedback logging module 50 receives and logs any edits of the report and/or feedback to the report provided by the clinician. The usage and feedback logging module 50 edits the logs (indicated by reference numeral 76) and provides the edits and/or feedback to a finetuning layer of a language model (as indicated by reference numeral 78) utilized by the question generation module 46 in generating questions for subsequently generate personalized patient reported outcome questionnaires. This enables accounting for any changes in language specific to a region. In particular, certain terms can be replaced with more regionally understood equivalent terms. The usage and feedback logging module 50 enables automatic learning from a doctor's, hospital's, or region's style of asking questions. In certain embodiments, the user profile and preferences 74 may be updated via the usage and feedback logging module 50 based on the received edits and/or feedback.

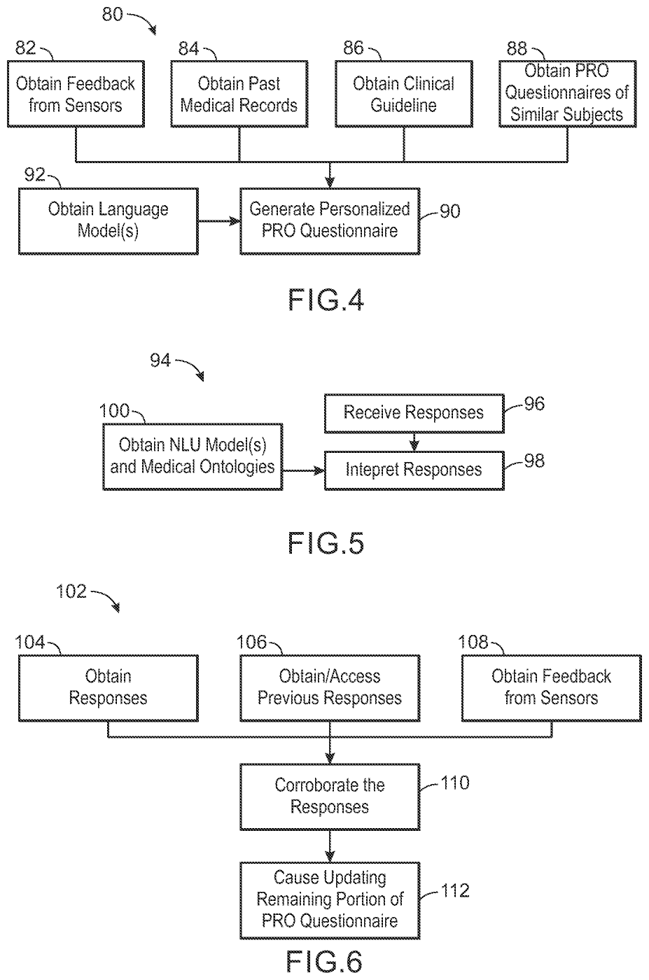

FIG. 4 is a flow chart of a method 80 for generating a personalized patient reported outcome (PRO) questionnaire. One or more steps of the method 80 may be performed by one or more components (e.g., processing system 38) of the remote patient monitoring system 12 in FIG. 1. One or more steps of the method 80 may be performed simultaneously and/or in a different order from that depicted in FIG. 4.

The method 80 includes obtaining feedback from one or more sensors, wherein the feedback relates to one or more health parameters of a subject (e.g., patient) (block 82). The method 80 also includes obtaining past medical records of the subject (block 84). In certain embodiments, the method 80 further includes obtaining a clinical guideline specific to a condition of the subject (block 86). In certain embodiments, the method 80 includes obtaining patient reported outcome (PRO) questionnaires of other subjects with a similar medical history or patient profile to the subject (block 88). The method 80 even further includes generating a personalized patient reported outcome questionnaire based at least on the feedback, the past medical records of the subject, and the clinical guideline, wherein the personalized patient reported outcome questionnaire is compatible with the clinical guideline (block 90). In certain embodiments, the patient reported outcome (PRO) questionnaires of other subjects with a similar medical history or patient profile to the subject are also utilized in generating the personalized patient reported outcome questionnaires. The method 80 also includes obtaining one or more language models (block 92) and utilizes the one or more language models in generating the questions for the personalized patient reported outcome questionnaire (block 90).

FIG. 5 is a flow chart of a method 94 for processing responses to a personalized patient reported outcome questionnaire. One or more steps of the method 94 may be performed by one or more components (e.g., processing system 38) of the remote patient monitoring system 12 in FIG. 1. One or more steps of the method 94 may be performed simultaneously and/or in a different order from that depicted in FIG. 5.

The method 94 includes receiving the answers or responses to questions (e.g., in a text form) from the subject (e.g., patient) to the personalized patient reported outcome questionnaire (block 96). The method 94 also includes interpreting the answers or responses to the questions from the subject to the personalized patient reported outcome questionnaire (block 98). The method 94 further includes obtaining one or more natural language understanding models and medical ontologies (block 100) and utilizing the natural language understanding (NLU) models and medical ontologies in interpreting the responses to questions from the subject (block 98). In certain embodiments, interpreting the answer or responses includes analyzing the one or more responses from the subject utilizing the natural language understanding model to determine any facts outside the scope of the personalized patient reported outcome questionnaire but relevant to subsequent clinical decisions.

FIG. 6 is a flow chart of a method 102 for corroborating responses to a personalized patient reported outcome questionnaire. One or more steps of the method 102 may be performed by one or more components (e.g., processing system 38) of the remote patient monitoring system 12 in FIG. 1. One or more steps of the method 102 may be performed simultaneously and/or in a different order from that depicted in FIG. 6.

The method 102 includes obtaining the responses to the questions from the questionnaire (e.g., from the verbal-response understanding module) (block 104). The method 102 also includes obtaining/accessing previous responses to the questionnaire by the patient and the medical records of the patient (e.g., patient profile, past complaint parameters, past diagnostic parameters, etc.). (block 106) The method 102 further includes obtaining the feedback from sensors of health or physiological parameters of the subject (e.g., patient) (block 108). The method 102 even further includes corroborating the responses with past responses to questions by the subject, the feedback from the sensors, and the past medical records of the subject (block 110). In certain embodiments, the response corroboration module 44 includes causing the updating of the remaining portion of the personalized patient reported outcome questionnaire based on a level of corroboration between the present responses and the past responses, the feedback, and the past medical records of the subject (block 112).

In certain embodiments, causing the updating includes notifying a question generation module when one or more of the present responses are not consistent with the past responses to the questions, the feedback, and/or the past medical records of the subject. In certain embodiments, causing the updating includes causing the question generation module to generate one or more follow-up questions to clarify any inconsistency when one or more of the present responses are not consistent with the past responses to the questions, the feedback, and/or the past medical records of the subject.

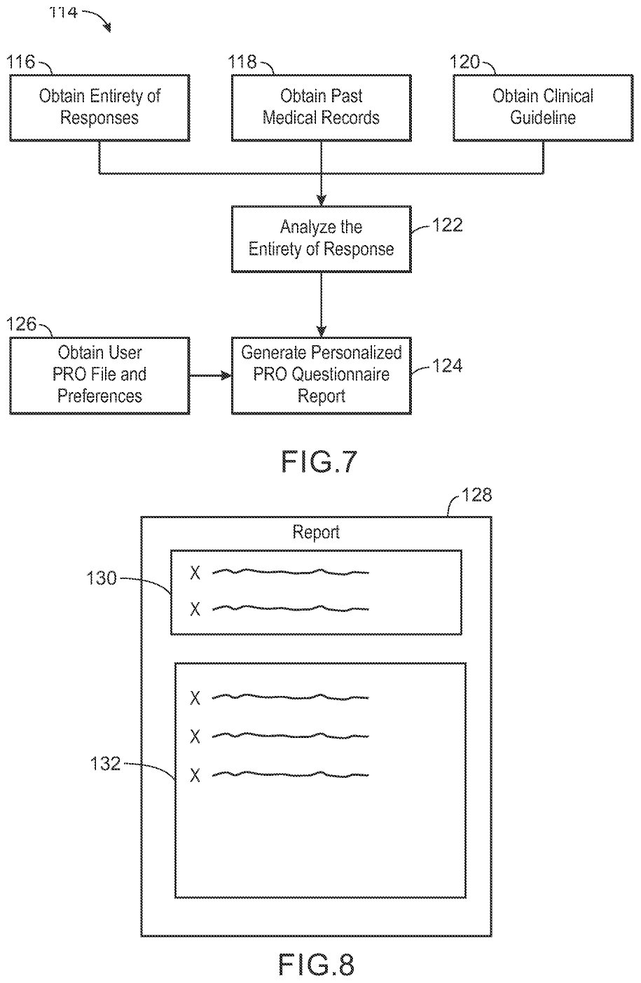

FIG. 7 is a flow chart of a method 114 for generating a personalized patient reported outcome questionnaire report. One or more steps of the method 114 may be performed by one or more components (e.g., processing system 38) of the remote patient monitoring system 12 in FIG. 1. One or more steps of the method 114 may be performed simultaneously and/or in a different order from that depicted in FIG. 7.

The method 114 obtaining an entirety of responses from the patient to the personalized patient reported outcome questionnaire (block 116). The method 114 also includes obtaining past medical records of the subject (e.g., patient profile, past complaint parameters, past diagnostic parameters, past treatment parameters, previous responses to patient reported outcome questionnaires, etc.) (block 118). The method 114 further includes obtaining a clinical guideline specific to a condition of the patient (block 120). The method 114 even further includes analyzing the entirety of the responses from the subject in the context of both the past medical records of the patient and the obtained clinical guideline (block 122). The method 114 still further includes generating a personalized patient reported outcome questionnaire report based on this analysis (block 124). Generating the personalized patient reported outcome questionnaire report includes ranking facts within the report in terms of relevancy and novelty and to present the facts in a concise manner. The most relevant and novel facts are presented at the beginning of the report. For example, when answers are beyond an expected range, or pertains to a new symptom or adverse event, can be highlighted. The method 114 includes obtaining user profile and user preferences for the clinician (block 126) and to utilize these in generating the report (block 124).

FIG. 8 is a schematic diagram of a personalized patient reported outcome questionnaire report 128. As noted above, the personalized patient reported outcome questionnaire report 128 includes the facts ranked within the report in terms of relevancy and novelty and presented in a concise manner. As depicted in FIG. 8, the report 128 includes the most relevant and novel facts presented at a beginning portion 130 of the report 128. The rest of fats are presented in a subsequent portion 132 of the report 128.

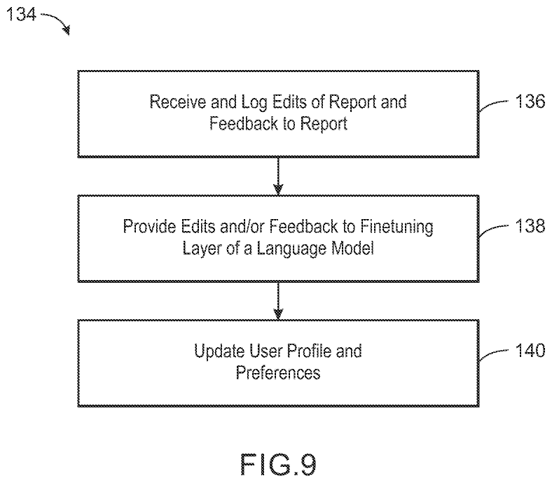

FIG. 9 is a flow chart of a method 134 for utilizing clinician edits and feedbacks. One or more steps of the method 134 may be performed by one or more components (e.g., processing system 38) of the remote patient monitoring system 12 in FIG. 1. One or more steps of the method 134 may be performed simultaneously and/or in a different order from that depicted in FIG. 9.

The method 134 includes receiving and logging any edits of the report and/or feedback to the report provided by the clinician (block 136). The method 134 also includes providing the edits and/or feedback to a finetuning layer of a language model utilized by the question generation module in generating questions for subsequently generate personalized patient reported outcome questionnaires (block 138). The method 134 further includes updating user profile and user preferences based on the edits and/or feedback (block 140).

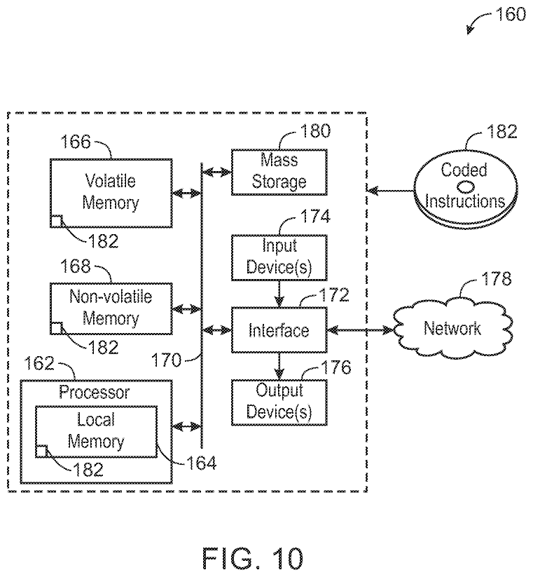

FIG. 10 is a block diagram of an example processor platform 160 for a portable computing device to be utilized with the disclosed techniques. The processor platform 160 of the illustrated example includes a processor 162. The processor 162 of the illustrated example is hardware. For example, the processor 162 can be implemented by integrated circuits, logic circuits, microprocessors or controllers from any desired family or manufacturer.

The processor 162 of the illustrated example includes a local memory 164 (e.g., a cache). The example processor 162 of FIG. 10 executes the instructions of at least FIGS. 4 and 5 to implement the systems, infrastructure, displays, and associated methods of FIGS. 4 and 5. The processor 162 of the illustrated example is in communication with a main memory including a volatile memory 166 and a non-volatile memory 168 via a bus 170. The volatile memory 166 may be implemented by Synchronous Dynamic Random Access Memory (SDRAM), Dynamic Random Access Memory (DRAM), RAMBUS Dynamic Random Access Memory (RDRAM) and/or any other type of random access memory device. The non-volatile memory 168 may be implemented by flash memory and/or any other desired type of memory device. Access to the main memory 166, 168 is controlled by a clock controller.

The processor platform 160 of the illustrated example also includes an interface circuit 172. The interface circuit 172 may be implemented by any type of interface standard, such as an Ethernet interface, a universal serial bus (USB), and/or a PCI express interface.

In the illustrated example, one or more input devices 174 are connected to the interface circuit 172. The input device(s) 174 permit(s) a user to enter data and commands into the processor 162. The input device(s) 174 can be implemented by, for example, a sensor, a microphone, a camera (still or video, RGB or depth, etc.), a keyboard, a button, a mouse, a touchscreen, a track-pad, a trackball, isopoint and/or a voice recognition system.

One or more output devices 176 are also connected to the interface circuit 172 of the illustrated example. The output devices 176 can be implemented, for example, by display devices (e.g., a light emitting diode (LED), an organic light emitting diode (OLED), a liquid crystal display, a cathode ray tube display (CRT), a touchscreen, a tactile output device, and/or speakers). The interface circuit 172 of the illustrated example, thus, typically includes a graphics driver card, a graphics driver chip or a graphics driver processor.

The interface circuit 172 of the illustrated example also includes a communication device such as a transmitter, a receiver, a transceiver, a modem and/or network interface card to facilitate exchange of data with external machines (e.g., computing devices of any kind) via a network 178 (e.g., an Ethernet connection, a digital subscriber line (DSL), a telephone line, coaxial cable, a cellular telephone system, Wi-Fi, etc.).

The processor platform 160 of the illustrated example also includes one or more mass storage devices 180 for storing software and/or data. Examples of such mass storage devices 180 include floppy disk drives, hard drive disks, compact disk drives, Blu-ray disk drives, RAID systems, and digital versatile disk (DVD) drives.

Coded instructions 182 may be stored in the mass storage device 180, in the volatile memory 166, in the non-volatile memory 168, and/or on a removable tangible computer readable storage medium (e.g., mass storage device 180).

Technical effects of the disclosed embodiments include providing for the digitization of guidelines (e.g., graphically) to enable the following of patient over time with respect to a condition. Technical effects of the disclosed embodiments also enable the digitized guidelines to adapt to any changes in the structure which enables the clinician (e.g. oncologist) keep up with the latest updates in the guidelines without any effort from their end. Technical effects of the disclosed embodiments include further helping find similar patients and questionnaires using patient histories and enable generating similar questionnaires for the patient of interest. Technical effects of the disclosed embodiments include enabling all elements of a patient's journey (and similar patient journeys) to be considered. Technical effects of the disclosed embodiments also include enabling more accurate questionnaires to be sent to remote patients using an algorithm that takes into account the rich patient data and history. Technical effects of the disclosed embodiments include enabling the improvement in access and speed and delivery of care to the patient. Technical effects of the disclosed embodiments include improving the efficiency and accuracy of remote patient monitoring and, thus, the quality of life of the patient. Technical effects of the disclosed embodiments also include reducing the burden on healthcare facilities and clinicians in generating the questionnaires. Technical effects of the disclosed embodiments further include reducing cost saving across healthcare.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A remote patient monitoring system, comprising:
a memory encoding processor-executable routines; and
a processing system configured to access the memory and to execute the processor-executable routines, wherein the processor-executable routines, when executed by the processing system, cause the processing system to:
obtain feedback from one or more sensors, wherein the feedback relates to one or more health parameters of a subject;
obtain past medical records of the subject;
obtain a clinical guideline specific to a condition of the subject; and
generate, via a question generation module, a personalized patient reported outcome questionnaire based at least on the feedback, the past medical records of the subject, and the clinical guideline, wherein the personalized patient reported outcome questionnaire is compatible with the clinical guideline.

2. The remote patient monitoring system of claim 1, wherein the processor-executable routines, when executed by the processing system, further cause the processing system to:
obtain questionnaires of other subjects with a similar medical history to the subject; and
generate, via the question generation module, the personalized patient reported outcome questionnaire based at least on the feedback, the past medical records of the subject, the clinical guideline, and the questionnaires of the other subjects.

3. The remote patient monitoring system of claim 1, wherein the processor-executable routines, when executed by the processing system, further cause the processing system to:
obtain, at a response understanding module, one or more responses from the subject to one or more questions from the personalized patient reported outcome questionnaire;
corroborate, via a response corroboration module, the one or more responses with past responses to questions by the subject, the feedback, and the past medical records of the subject; and
update, via the question generation module, a remaining portion of the personalized patient reported outcome questionnaire based on a level of corroboration between the one or more responses with the past responses to questions by the subject, the feedback, and the past medical records of the subject.

4. The remote patient monitoring system of claim 3, wherein the processor-executable routines, when executed by the processing system, further cause the processing system to:
notify, via the response corroboration module, the question generation module when the one or more responses are not consistent with the past responses to the questions by the subject, the feedback, and/or the past medical records of the subject; and
generate, via the question generation module, one or more follow-up questions to clarify any inconsistency when the one or more responses are not consistent with the past responses to the questions by the subject, the feedback, and/or the past medical records of the subject.

5. The remote patient monitoring system of claim 1, wherein the processor-executable routines, when executed by the processing system, further cause the processing system to:
obtain, at a response understanding module, one or more responses from the subject to one or more questions from the personalized patient reported outcome questionnaire, wherein the one or more responses are outside a scope of the personalized patient reported outcome questionnaire but relevant to subsequent clinical decisions; and
generate, via the question generation module, one or more follow-up questions based on the one or more responses.

6. The remote patient monitoring system of claim 5, wherein the processor-executable routines, when executed by the processing system, further cause the processing system to:
analyze, via the response understanding module, the one or more responses from the subject utilizing a natural language understanding model to determine any facts outside the scope of the personalized patient reported outcome questionnaire but relevant to subsequent clinical decisions.

7. The remote patient monitoring system of claim 1, wherein the processor-executable routines, when executed by the processing system, further cause the processing system to:
receive, at a report generation module, an entirety of responses from the subject to the personalized patient reported outcome questionnaire;
analyze, via the report generation module, the entirety of the responses from the subject in context of both the past medical records of the subject and the clinical guideline; and
generate, via the report generation module, a personalized patient reported outcome questionnaire report based on the analysis, wherein facts within the personalized patient reported outcome questionnaire report are ranked in terms of relevancy and novelty and presented in a concise manner.

8. The remote patient monitoring system of claim 7, wherein the processor-executable routines, when executed by the processing system, further cause the processing system to:
log, via a usage and feedback logging module, edits to the personalized patient reported outcome questionnaire report; and
provide, via the usage and feedback logging module, the edits to a finetuning layer of a language model utilized by the question generation module in generating questions for subsequently generated personalized patient reported outcome questionnaires.

9. A computer-implemented method for remote patient monitoring, comprising:
obtaining, via a processing system, feedback from one or more sensors, wherein the feedback relates to one or more health parameters of a subject;
obtaining, via the processing system, past medical records of the subject;
obtaining, via the processing system, a clinical guideline specific to a condition of the subject; and
generating, via a question generation module of the processing system, a personalized patient reported outcome questionnaire based at least on the feedback, the past medical records of the subject, and the clinical guideline, wherein the personalized patient reported outcome questionnaire is compatible with the clinical guideline.

10. The computer-implemented method of claim 9, further comprising:
obtaining, at the processing system, questionnaires of other subjects with a similar medical history to the subject; and
generating, via the question generation module of the processing system, the personalized patient reported outcome questionnaire based at least on the feedback, the past medical records of the subject, the clinical guideline, and the questionnaires of the other subjects.

11. The computer-implemented method of claim 9, further comprising:
obtaining, at a response understanding module of the processing system, one or more responses from the subject to one or more questions from the personalized patient reported outcome questionnaire;
corroborating, via a response corroboration module of the processing system, the one or more responses with past responses to questions by the subject, the feedback, and the past medical records of the subject; and
updating, via the question generation module of the processing system, a remaining portion of the personalized patient reported outcome questionnaire based on a level of corroboration between the one or more responses with the past responses to questions by the subject, the feedback, and the past medical records of the subject.

12. The computer-implemented method of claim 11, further comprising:
notifying, via the response corroboration module of the processing system, the question generation module when the one or more responses are not consistent with the past responses to the questions by the subject, the feedback, and/or the past medical records of the subject; and
generating, via the question generation module of the processing system, one or more follow-up questions to clarify any inconsistency when the one or more responses are not consistent with the past responses to the questions by the subject, the feedback, and/or the past medical records of the subject.

13. The computer-implemented method of claim 9, further comprising:
obtaining, at a response understanding module of the processing system, one or more responses from the subject to one or more questions from the personalized patient reported outcome questionnaire, wherein the one or more responses are outside a scope of the personalized patient reported outcome questionnaire but relevant to subsequent clinical decisions; and
generating, via the question generation module of the processing system, one or more follow-up questions based on the one or more responses.

14. The computer-implemented method of claim 13, further comprising:
analyzing, via the response understanding module of the processing system, the one or more responses from the subject utilizing a natural language understanding model to determine any facts outside the scope of the personalized patient reported outcome questionnaire but relevant to subsequent clinical decisions.

15. The computer-implemented method of claim 9, further comprising:
receiving, at a report generation module of a processing system, an entirety of responses from the subject to the personalized patient reported outcome questionnaire;
analyzing, via the report generation module of a processing system, the entirety of the responses from the subject in context of both the past medical records of the subject and the clinical guideline; and
generating, via the report generation module of the processing system, a personalized patient reported outcome questionnaire report based on the analysis, wherein facts within the personalized patient reported outcome questionnaire report are ranked in terms of relevancy and novelty and presented in a concise manner.

16. The computer-implemented method of claim 15, further comprising:
logging, via a usage and feedback logging module of the processing system, edits to the personalized patient reported outcome questionnaire report; and
providing, via the usage and feedback logging module of the processing system, the edits to a finetuning layer of a language model utilized by the question generation module in generating questions for subsequently generated personalized patient reported outcome questionnaires.

17. A non-transitory computer-readable medium, the computer-readable medium comprising processor-executable code that when executed by a processing system, causes the processing system to:
obtain feedback from one or more sensors, wherein the feedback relates to one or more health parameters of a subject;
obtain past medical records of the subject;
obtain a clinical guideline specific to a condition of the subject; and
generate, via a question generation module, a personalized patient reported outcome questionnaire based at least on the feedback, the past medical records of the subject, and the clinical guideline, wherein the personalized patient reported outcome questionnaire is compatible with the clinical guideline.

18. The non-transitory computer-readable medium of claim 17, wherein the processor-executable code, when executed by the processing system, further causes the processing system to:
obtain, at a response understanding module, one or more responses from the subject to one or more questions from the personalized patient reported outcome questionnaire;
corroborate, via a response corroboration module, the one or more responses with past responses to questions by the subject, the feedback, and the past medical records of the subject; and
update, via the question generation module, a remaining portion of the personalized patient reported outcome questionnaire based on a level of corroboration between the one or more responses with the past responses to questions by the subject, the feedback, and the past medical records of the subject.

19. The non-transitory computer-readable medium of claim 17, wherein the processor-executable code, when executed by the processor, further causes the processing system to:
obtain, at a response understanding module, one or more responses from the subject to one or more questions from the personalized patient reported outcome questionnaire, wherein the one or more responses are outside a scope of the personalized patient reported outcome questionnaire but relevant to subsequent clinical decisions; and
generate, via the question generation module, one or more follow-up questions based on the one or more responses.

20. The non-transitory computer-readable medium of claim 17, wherein the processor-executable code, when executed by the processor, further causes the processing system to:
receive, at a report generation module, an entirety of responses from the subject to the personalized patient reported outcome questionnaire;
analyze, via the report generation module, the entirety of the responses from the subject in context of both the past medical records of the subject and the clinical guideline; and
generate, via the report generation module, a personalized patient reported outcome questionnaire report based on the analysis, wherein facts within the personalized patient reported outcome questionnaire report are ranked in terms of relevancy and novelty and presented in a concise manner.
